(19) United States
(12) Patent Application Publication
(10) Pub. No.: US 2018/0037604 A1
(43) Pub. Date: Feb. 8, 2018

(54) PROCESS FOR THE PURIFICATION OF RECOMBINANT PROTEINS

(71) Applicant: STELIS BIOPHARMA PRIVATE LIMITED, Bangalore (IN)

(72) Inventors: Rajanarendra Reddy GUNTHAKAL, Bangalore (IN); Madhava Reddy GONGATI, Bangalore (IN); Durgaprasad ANNAVAJJULA, Bangalore (IN)

(73) Assignee: Stelis Biopharma Private Limited

(21) Appl. No.: 15/271,472

(22) Filed: Sep. 21, 2016

(30) Foreign Application Priority Data
Aug. 4, 2016 (IN) 201641026646

(51) Int. Cl. C07K 1/36; C07K 14/565; B01D 11/04; B01D 15/36; B01D 61/14; C07K 1/14; C07K 14/62

(52) U.S. Cl. CPC C07K 1/36; C07K 1/145; C07K 14/565; C07K 14/62; B01D 15/362; B01D 61/145; B01D 11/0492; B01D 15/363; C07K 2319/00; B01D 2311/2634; B01D 2311/2623; B01D 2315/16

(57) ABSTRACT

A process for the extraction and purification of recombinant proteins, more specifically interferons and insulin, having improved quality and yield. The process comprises extraction of proteins from bacterial inclusion bodies using an extraction solution comprising at least one detergent; at least one chaotropic agent; at least one cosmotropic agent; and a protein folding agent.

### TECHNICAL FIELD OF THE INVENTION

The present invention relates to an improved process for recombinant protein recovery. More particularly, the present invention relates to a novel process for the extraction and purification of interferon, insulin and its analogues, and related proteins such as the Insulin Growth Factor Binding Protein (IGF) in substantially pure form having improved quality and yield.

### BACKGROUND

Increased applications of recombinant proteins, including mammalian proteins in clinical and biochemical fields have resulted in a surge in their demand. Recombinant proteins have been utilized as tools for cellular and molecular biology. In the course of heterologous recombinant protein production, efficient protein expression rates are essential. However, rapid intracellular protein accumulation and expression of large proteins increases the probability of aggregation. During the various stages of recombinant protein production and phases of fermentation, protein purification, and long-term storage protein aggregation is likely to occur. Misfolded proteins which accumulate in intracellular aggregates are known as inclusion bodies. Inclusion body formation is likely to occur during production of recombinant proteins, rendering it difficult and expensive to obtain soluble functional protein fraction post downstream operations. Downstream processing stages of recombinant proteins synthesized in bacterial host expression systems serve as an important pre-requisite in the retention and preservation of protein conformity, structural integrity and activity. Several recombinant proteins are expressed in a misfolded form and as aggregates in host expression systems, generally in the form of bacterial inclusion bodies. The protein aggregates can cause structural strain to the cell, are toxic and have been implicated in amyloidoses, including Amyotrophic lateral sclerosis (ALS), Alzheimer's, Parkinson's and prion disease. Therefore, extraction and purification stages involve the use of renaturing agents to achieve appropriate protein folding.

Moreover, the cost involved in protein purification is observed to be significantly higher. Rosen et al. (Bio/Technol 11, 349-357) have studied that the overall costs for producing tissue plasminogen activator by expression in E. coli is greater than that for a mammalian cell-based bioprocess as a result of the higher expenses incurred during the solubilization and renaturation steps required in the recombinant DNA technology.

Deamidation of asparagine residues is one of the most common post-translational modifications occurring in therapeutic proteins produced using recombinant DNA technology. A reduction or loss of in vitro or in vivo biological activity has been reported for variety of proteins including recombinant human DNAse (Cacia et al., J. Chromatogr. 1993, 634:229-239) interferon beta (IFN-β) and recombinant soluble CD4 (Teshima et al., Biochemistry 1991, 30:3916-3922). It is therefore important to establish methods for characterizing sites of deamidation as well as for evaluating effect on biological activity and antigenicity (Glen Teshima, Deamidation in Proteins and Peptides, November-2000, Ion Source).

Interferon-β is one such protein which is found sequestered in inclusion bodies. Interferon-β for use in clinical and therapeutic studies must have relatively high purity and be substantially uncontaminated with toxic host cell constituents, cell debris, and other extraneous chemicals introduced during the extraction and purification steps. Commercially available IFN-β has several related impurities comprising deamidated forms of proteins and aggregates upto 40% and 10% respectively of the final product. Conventional methods employed involve tedious scale up and production stages and harsh detergents and organic solvents. Therefore, several methods for the preparation, recovery, and purification of IFN-β are prevalent in the art. IFN-β 1b is indicated for the treatment of relapsing forms of multiple sclerosis to reduce the frequency of clinical exacerbations. Commercially, IFN-β has been manufactured either in CHO cells in the soluble form or in the form of inclusion bodies expressed in host cells such as E. coli or Pseudomonas. However, post expression of the proteins, the downstream process stages involve physical methods to obtain substantially pure IFN-β using harsh treatment procedures such as subjection to high pressure, microwave assistance, solubilization of proteins (Datta et al. Sustainable Chemical Processes 2013, 1:2) and agitation. Several extraction procedures including the use of Guanidine-HCl, urea, pH have been described in previous literature, these treatment processes though being harsh are not effective in the solubilization of tough proteins like interferons, thereby resulting in lower yields and higher product related impurities.

U.S. Pat. No. 8,273,561 utilizes high pressure greater than 3000 bars to treat aggregated interferons, particularly recombinant human interferon-β, to reduce the aggregate content of interferon material. It also mentions the use of a refolding agent such as arginine; however its incorporation does not bring about any improved yield in the refolding of proteins. The application of high pressure to the aggregated interferons may result in the loss of functional properties of the final product.

US2011217784 relates to the extraction of soluble, active recombinant type 1 interferon protein from an insoluble fraction without the use of denaturation and without the need for a refolding step. Employing the process described therein resulted in decreased protein recovery and lower purity levels.

The refolding stage of several purification processes is slow with overnight stirring and agitation, thereby increasing process time, costs and lowering product quality and yield.

Conventional extraction methods yield a minimal recovery of 2% of the protein of interest and involve the application of harsh conditions including the use of reagents such as sodium dodecyl sulphate (SDS) and butanol.

Therefore, the present inventors have provided an extraction solution and the use of the same in the extraction and purification of recombinant proteins.

### OBJECTS OF THE INVENTION

The defining object of the present invention is to provide an extraction solution for recovery of recombinant proteins sequestered in bacterial inclusion bodies.

A further object of the present invention is to provide a process for the recovery and purification of recombinant proteins so as to increase the solubilization, quality and yield of the end product.

Another object of the present invention is to provide a pharmaceutical formulation comprising the recombinant protein recovered and purified by the instant process.

### SUMMARY OF THE INVENTION

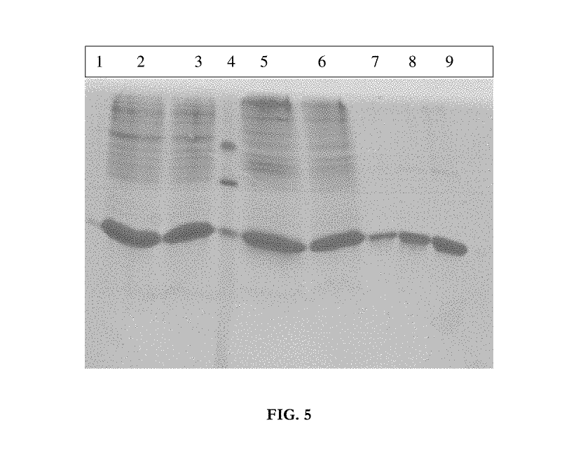

The present invention discloses a process for the recovery and purification of recombinant proteins sequestered in inclusion bodies, the said method comprising dissolution of protein aggregates, reduction in the aggregation (less than 2%) and deamidation (FIGS. 5(a) and 5(b)) thereby providing recovery of recombinant proteins in increased yield and having improved product quality.

In various embodiments, the present invention provides a process for the recovery and purification of a recombinant protein from bacterial inclusion bodies comprising,
i. lysing host cells containing said protein to obtain a cell lysate followed by centrifuging the lysate to obtain an insoluble fraction,
ii. subjecting the said insoluble fraction to treatment with an extraction solution comprising; a) at least one detergent, b) at least one chaotropic agent, c) at least one cosmotropic agent, and d) at least one protein folding agent, wherein the extraction solution has a pH of around 1.5 to around 12, to obtain a solubilized fraction,
iii. allowing the solubilized fraction to mature followed by addition of an oxidizing agent to the extraction mixture, and
iv. filtering the extract and subjecting it to ion exchange chromatography followed by ultrafiltration and diafiltration to obtain an extract comprising said protein in soluble form, in high yield with high % purity.

In some embodiments, the chaotropic agent may be, but is not limited to, urea, guanidinium chloride, thiourea, or a mixture thereof. The cosmotropic agent may be, but is not limited to, sodium chloride, potassium chloride, ammonium sulfate, or a mixture thereof. The protein folding agent may be arginine. The detergent and the chaotropic agent may have an alkaline pH.

In some aspects, the present disclosure provides a process for the recovery and purification of a recombinant protein from bacterial inclusion bodies comprising,
i. lysing host cells containing said protein to obtain a cell lysate followed by centrifuging the lysate to obtain an insoluble fraction,
ii. subjecting the said insoluble fraction to treatment with an extraction solution comprising; a) at least one detergent having alkaline pH, b) at least one chaotropic agent having alkaline pH, c) at least one cosmotropic agent and d) at least one protein folding agent viz. arginine, wherein, the extraction solution has a pH of around 1.5 to around 12, to obtain a solubilized fraction,
iii. allowing the solubilized fraction to mature followed by addition of an oxidizing agent to the extraction mixture, and
iv. filtering the extract and subjecting it to ion exchange chromatography followed by ultrafiltration and diafiltration to obtain an extract comprising said protein in soluble form, in high yield with high % purity.

Accordingly, the present disclosure provides a process for recovery and purification of recombinant proteins expressed intracellularly in bacterial inclusion bodies such as a recombinant Type 1 interferon protein selected from the group consisting of interferon-β, interferon-α, interferon-κ, interferon-τ, or an interferon-ω as well as insulin and its analogues selected from glargine, lis-proinsulin and aspart insulin, and insulin like growth factor binding protein (IGF).

In some embodiments, the present disclosure provides a process for recovery and purification of recombinant proinsulin fusion proteins, using an extraction solution comprising at least one detergent having an alkaline pH, at least one chaotropic agent having alkaline pH, c) at least one cosmotropic agent and d) at least one protein folding agent, e.g., arginine, wherein the extraction solution has a pH of around 1.5 to around 12. The recombinant proinsulin fusion protein is a fusion protein comprising a proinsulin peptide chain linked to a second biologically active peptide chain. The second biologically active peptide chain may be transferrin, interleukin-2, and glutathione-S-oxidase.

In an aspect, the present disclosure provides a process for the recovery and purification of a recombinant type 1 interferon from bacterial inclusion bodies comprising, lysing host cells containing said protein to obtain a cell lysate followed by centrifuging the cell lysate to obtain an insoluble fraction,
i. subjecting the said insoluble fraction to treatment with an extraction solution comprising; a) at least one detergent having alkaline pH, b) at least one chaotropic agent having alkaline pH, c) at least one cosmotropic agent and d) at least one protein folding agent viz. arginine, wherein the extraction solution has a pH of around 7 to around 8.5, to obtain a solubilized fraction,
ii. allowing the solubilized fraction to mature followed by addition of an oxidizing agent to the extraction mixture, and
iii. filtering the extract and subjecting it to ion exchange chromatography followed by ultrafiltration and diafiltration to obtain an extract comprising said protein in soluble form, in high yield with high % purity.

In another aspect, the present disclosure provides a process for the recovery and purification of recombinant insulin and analogues thereof from bacterial inclusion bodies comprising,
i. lysing host cells containing said protein to obtain a cell lysate followed by centrifuging the cell lysate to obtain an insoluble fraction,
ii. subjecting the said insoluble fraction to treatment with an extraction solution comprising; a) at least one detergent having alkaline pH, b) at least one chaotropic agent having alkaline pH, c) at least one cosmotropic agent and d) at least one protein folding agent viz. arginine,
   wherein, the extraction solution has a pH of around 1.5 to around 12, to obtain a solubilized fraction,
iii. allowing the solubilized fraction to mature followed by addition of an oxidizing agent to the extraction mixture, and
iv. filtering the extract and subjecting it to ion exchange chromatography followed by ultrafiltration and diafiltration to obtain an extract comprising recombinant protein in soluble form, in high yield with high % purity.

Insulin related proteins such as the Insulin Growth Factor Binding Protein (IGF) expressed in bacterial inclusion bodies may also be recovered and purified by the present process.

In another aspect, the present disclosure provides an extraction solution employed in the protein recovery and purification process, said solution comprising
a) at least one detergent having alkaline pH,
b) at least one chaotropic agent having alkaline pH,
c) at least one cosmotropic agent and
d) at least one protein folding agent viz. arginine,
   wherein, the extraction solution has a pH of around 1.5 to around 12.

In one more aspect, the present disclosure provides a kit for recovery of proteins from bacterial inclusion bodies, comprising:
(i) an extraction solution for dissolving said proteins, comprising at least one detergent having alkaline pH; at least one chaotropic agent having alkaline pH; at least one cosmotropic agent; and arginine; and
(ii) an oxidizing agent selected from thiol, a disulfide, or a mixture thereof for oxidizing said dissolved proteins;
   wherein said extraction solution has a pH of between about 1.5 and about 12.

### DETAILED DESCRIPTION OF FIGURES

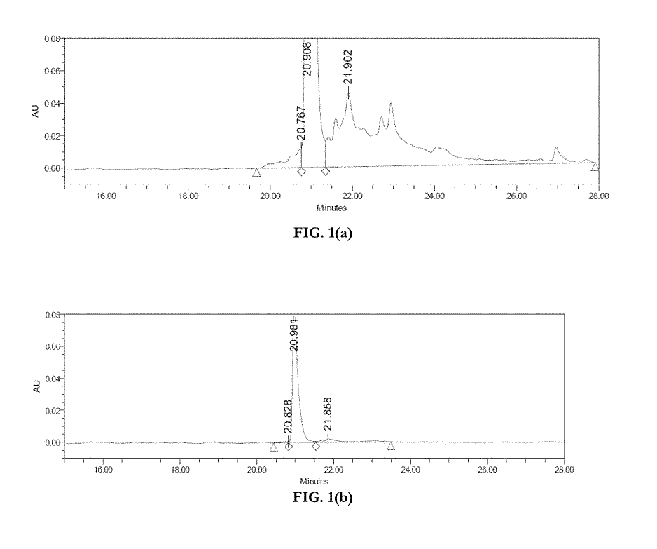

FIG. 1(a) depicts the 64% purity of pro-insulin obtained by HPLC studies at the refolding stage;

FIG. 1(b) depicts around 90% purity of pro-insulin at the final purification stage. Pro-insulin was extracted by solubilization of the protein at 1:24 ratio of the inclusion bodies to the extraction buffer in the presence of arginine;

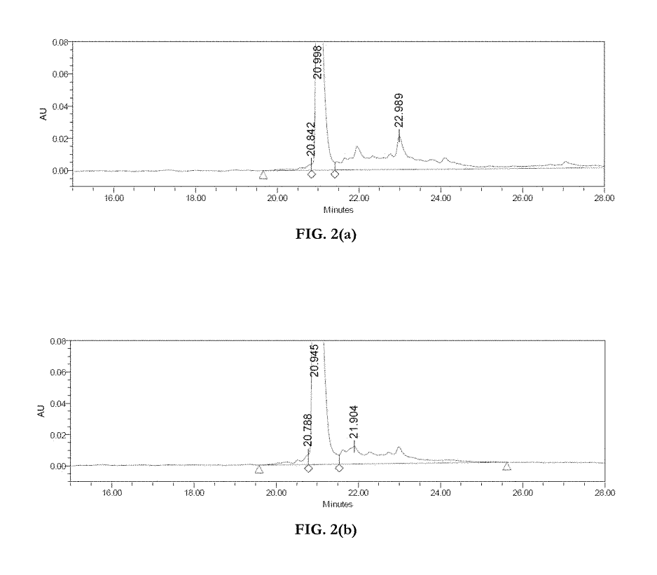

FIG. 2(a) depicts the 54.9% purity of pro-insulin obtained by HPLC studies at the refolding stage; and

FIG. 2(b) depicts around 85% purity of pro-insulin at the final purification stage. Pro-insulin was extracted by solubilization of the protein at 1:24 ratio of the inclusion bodies to the extraction buffer in the absence of arginine.

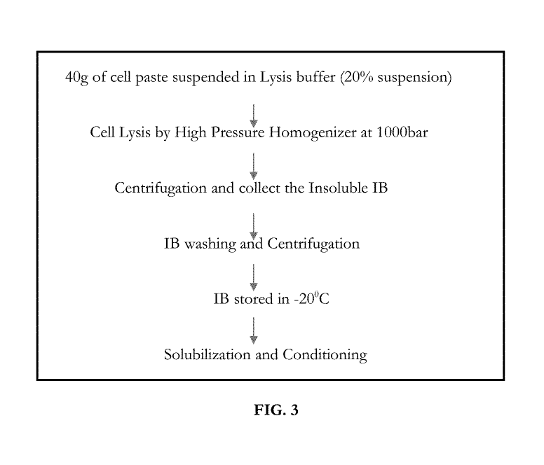

FIG. 3 depicts a schematic representation of the process for purification of IFN β1b as disclosed herein;

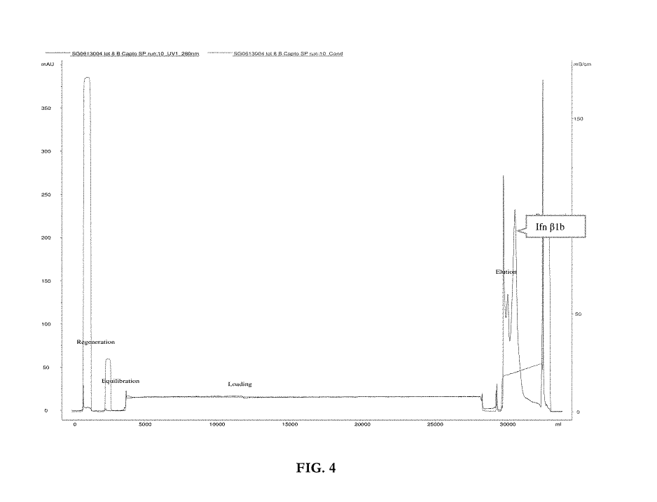

FIG. 4 is a cation exchange chromatogram determining the concentration of interferon-β 1b content measured at absorbance of 280 nm, loaded on 225 ml column resin;

FIG. 5 depicts the SDS PAGE analysis for IFNβ 1b; Read from left to right: Lane 1 refers to the Blank, lane 2: sample subjected to extraction with arginine in the a reduced buffer, lane 3: Protein sample extracted in the absence of arginine; lane 4: Marker; lane 5: Sample extracted with arginine in a non-reduced buffer, lane 6: sample extracted without arginine in a non-reduced buffer, Lanes 7 to 9 have dodecyl sulphate (DS) run in varying concentrations, and in Lane 10 a blank is run;

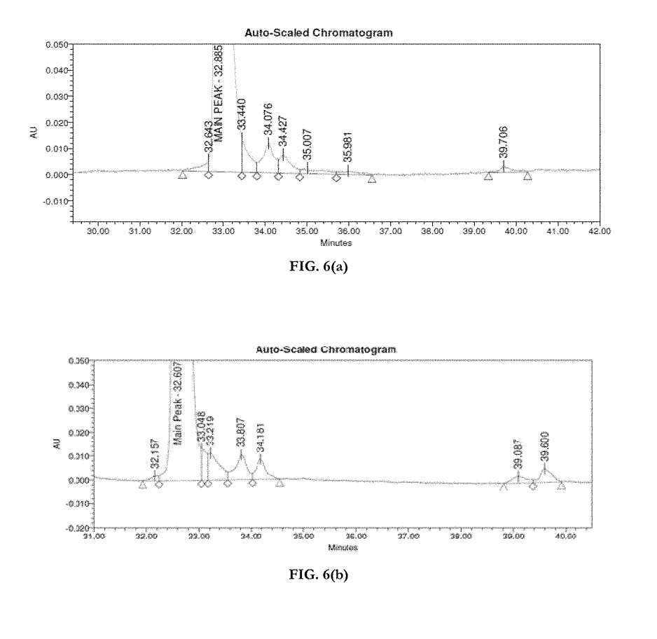

FIG. 6(a) depicts the RP HPLC profile of maturation of recombinant interferon β 1b without addition of an oxidizing agent after CEX pooled elution;

FIG. 6(b) depicts the RP HPLC profile of maturation of recombinant interferon β 1b with an oxidizing agent after CEX pooled elution;

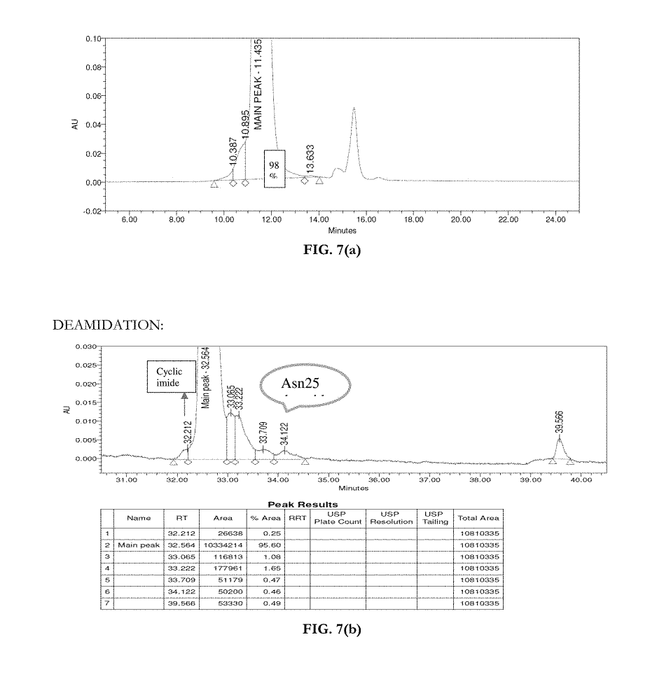

FIG. 7(a) and FIG. 7(b) depicts dissolution of protein aggregates and reduction in the aggregation (less than 2%) and deamidation according to the purification method;

### DETAILED DESCRIPTION

The invention will now be described in detail in connection with certain preferred and optional embodiments, so that various aspects thereof may be more fully understood and appreciated.

The present disclosure provides a process for purification of recombinant proteins sequestered in bacterial inclusion bodies, the said method involving dissolution of protein aggregates and reduction in the deamidation limit, thereby providing enhanced recovery of the recombinant protein in increased yield and having improved product quality. In the most preferred embodiment, the present disclosure provides a process for the recovery and purification of a recombinant protein from bacterial inclusion bodies comprising,
i. lysing host cells containing said protein to obtain a cell lysate followed by centrifuging the lysate to obtain an insoluble fraction,
ii. subjecting the said insoluble fraction to treatment with an extraction solution comprising; a) at least one detergent having alkaline pH, b) at least one chaotropic agent having alkaline pH, c) at least one cosmotropic agent and d) at least one protein folding agent viz. arginine, wherein, the extraction solution has a pH of around 1.5 to around 12, to obtain a solubilized fraction,
iii. allowing the solubilized fraction to mature followed by addition of an oxidizing agent to the extraction mixture, and
iv. filtering the extract and subjecting it to ion exchange chromatography followed by ultrafiltration and diafiltration to obtain an extract comprising said protein in soluble form, in high yield with high % purity.

The recombinant protein to be recovered and purified from bacterial inclusion bodies is expressed in a suitable bacterial host cell, the said host cell is selected from E. coli, Bacillus, Pseudomonas species, etc, which are capable of expressing the proteins through recombinant technology. The host cell comprises an expression vector carrying a nucleotide sequence encoding the said recombinant protein. Protein expression is induced and cells are lysed by high pressure homogenizing to release a cell lysate containing a soluble and insoluble fraction. A recombinant protein when synthesized in the form of aggregates in inclusion bodies, it is preferable to retain the insoluble fraction obtained on lysing of cells. The insoluble fraction is retained and washed with a buffer, pH 8. The washed fraction is further treated with an extraction solution. Around 70 ml of an extraction solution is added to each gram of the washed recombinant protein by wet weight. The solution is stirred continuously for one hour and then centrifuged. The supernatant obtained is reverse diluted and the solution is matured for 16 to 18 hours followed by addition of an oxidizing agent to the matured solution. The resultant solution is incubated for an hour and pH adjusted to 7.2. Post maturation, the solution is filtered through a membrane and sequentially subjected to cation exchange chromatography and later to anion exchange chromatography respectively. More specifically, the solution is loaded onto a cation-exchange column, followed by dilution and then passed onto an anion-exchange column. The eluate obtained is subjected to ultrafiltration and diafiltration (UFDF) processes, and the % purity of the UFDF retentate is estimated to be in the range of 92% to 97% (FIG. 3 describes the process for recovery and purification of recombinant protein). In accordance with the most preferred embodiment, the extraction solution employed in the protein recovery and purification process, said solution comprising
a) at least one detergent having alkaline pH,
b) at least one chaotropic agent having alkaline pH,
c) at least one cosmotropic agent and
d) at least one protein folding agent viz. arginine,
   wherein, the extraction solution has a pH of around 1.5 to around 12.

Accordingly, the chaotropic agent is selected from the group consisting of urea, guanidium chloride, thiourea and mixtures thereof. The detergent is selected from sulfobetaines.

The cosmotropic agent is selected from the group consisting of sodium chloride, potassium chloride, ammonium sulfate, and mixtures thereof.

Arginine is the protein folding agent used in the present process helps facilitate the folding of recombinant proteins that are sequestered in inclusion bodies, thus enabling the protein to attain a characteristic three-dimensional confirmation to perform its functions. The concentration of arginine employed as a protein folding agent in the present disclosure is in the range of 0.1M to 2 M. Arginine helps stabilize the protein when active disulphide exchange is performed in the consequent stages of the process.

Further, the oxidizing agent is selected from the group consisting of cystine, cysteine, or Glutathione (GSSG), Glutathione (GSH) and with or without the addition of DTT (Dithiothreitol).

In an embodiment, the process for recovery and purification of recombinant proteins expressed intracellularly in bacterial inclusion bodies such as a recombinant Type 1 interferon protein selected from the group consisting of interferon-β, interferon-α, interferon-κ, interferon-τ, or an interferon-ω as well as insulin and its analogues selected from pro-insulin, glargine, lis-proinsulin and aspart insulin, and insulin like growth factor binding protein (IGF).

In another preferred embodiment, the present disclosure provides a process for the recovery and purification of a recombinant type 1 interferon from bacterial inclusion bodies comprising, lysing host cells containing said protein to obtain a cell lysate followed by centrifuging the cell lysate to obtain an insoluble fraction,
i. subjecting the said insoluble fraction to treatment with an extraction solution comprising; a) at least one detergent having alkaline pH, b) at least one chaotropic agent having alkaline pH, c) at least one cosmotropic agent and d) at least one protein folding agent viz. arginine,
wherein, the extraction solution has a pH of around 7.2 to around 8.5, to obtain a solubilized fraction,
ii. allowing the solubilized fraction to mature followed by addition of an oxidizing agent to the extraction mixture, and
iii. filtering the extract and subjecting it to ion exchange chromatography followed by ultrafiltration and diafiltration to obtain an extract comprising said protein in soluble form, in high yield with high % purity.

The recombinant type 1 interferon selected from the group consisting of interferon-β, interferon-β 1b, interferon-α interferon-κ, interferon-τ and interferon-ω is recovered and purified by the present process.

In accordance with this preferred embodiment, recombinant interferon β-1b (IFNβ-1b) is expressed intracellularly in a suitable host cell, the host cell being selected from E. coli and Pseudomonas species. The host cell comprises an expression vector carrying a nucleotide sequence encoding the recombinant protein, i.e. IFN β-1b. The expression of the protein is induced and cells are lysed by high pressure homogenizing to release a cell lysate containing a soluble and insoluble fraction. Recombinant IFN β-1b is synthesized in the form of aggregates in inclusion bodies, therefore lysing of cells would result in the retention of IFN β-1b in the insoluble fraction. The insoluble fraction is retained and washed using Tris buffer, pH 8. The washed fraction is further treated with an extraction buffer. The extraction solution comprises Tris buffer, a cosmotropic agent, a chaotropic agent and arginine, the pH of the solution is maintained at 8. Around 70 ml of an extraction buffer is added to each gram of the washed IFN-β-1b by wet weight. The solution is stirred continuously for one hour and then centrifuged. The supernatant obtained is reverse diluted and the solution is matured for 16-18 hrs followed by addition of an oxidizing agent to the matured solution. The resultant solution is incubated for an hour and the pH is adjusted to 7.2.

The maturation of the recombinant β 1b with an oxidizing agent and in its absence is depicted in FIG. 6(a) and FIG. 6(b). FIG. 6(b) indicates that the reduced peaks adjoining the main peak are converted to the main peak when treated with the oxidizing agent. Thereby, helping in reducing the pre-peak as described in FIG. 6(a).

Post maturation, the solution is filtered through a membrane and then sequentially subjected to cation exchange chromatography and later to anion exchange chromatography respectively. More specifically, the solution is loaded onto a cation-exchange column, followed by dilution and then passed onto an anion-exchange column. The eluate obtained, is subjected to ultrafiltration and diafiltration processes, the % purity of the UFDF retentate is in the range of 92% to 98% with total protein recovery in the range of 185 to 195 mg.

Maintaining an alkaline pH of the solution throughout the purification process has a positive impact when moving from near neutral pH to slightly alkaline pH where active disulphide exchange results in great improvement in the product recovery. In the present disclosure, the extraction of the insoluble form of IFNβ-1b comprising selecting the concentration of the L-Arginine an maintaining the pH between 7.2-8.5 yields an overall product which is enhanced by 10 fold compared to processes which do not include L-arginine in the extraction solution and wherein maturation does not occur at alkaline pH.

The overall quality of the product obtained by the present process is improved by ˜10% as indicated by reverse phase chromatography results. The synergistic effect in improving the overall recovery and purity is expected to be because of enhanced solubilization/disaggregation by L-Arginine and active disulphide exchange at alkaline pH.

In another embodiment, the present disclosure provides that the cation exchange resin is selected from the group consisting of sepharose, cross-linked poly[styrene-co-divinylbenzene] with polyhydroxyl surface coating and high flow agarose columns.

More preferably, high flow agarose column such as the capto Impres column is employed for the instant purification process.

In another embodiment, the present process provides a process for the purification of IFN-β 1b, wherein the % purity of the end product is in the range of 92 to 97%. (FIG. 5 depicts the run of the extracted protein in lanes 2 and 5)

The % recovery and the purity of interferon beta 1b is determined at each stage of the process, i.e. post lysis, after cation exchange chromatography, after anion exchange chromatography and after ultrafiltration and diafiltration.

The total protein content post lysis is in the range of 700 to 750 mg, the % step recovery and % purity is not determined. After subjecting the cell lysate to extraction and maturation to obtain a solubilized fraction of IFN-β 1b, the solution is subjected to cation exchange chromatography, the total protein content is in the range of 220 to 250 mg with % recovery in the range of 30 to 40%, and % purity in the range of 90 to 95%.

After anion exchange chromatography, the total protein recovered is in the range of 200 to 215 mg, with recovery in the range of 90 to 95% and % purity in the range of 93% to 98%.

After the ultrafiltration and diafiltration stage, the total protein recovered is in the range of 180 to 195 mg, with % recovery in the range of 90 to 95% and % purity in the range of 95 to 98% (Table 6). The recombinant protein obtained by the present process was analyzed on an HPLC analytical column, and found to be 95.6% pure, after the final stage of ultrafiltration and diafiltration (Table 6, FIG. 7(b)), with a low content of deamidated products. The results of FIGS. 7(a) and 7(b) were obtained using analytical HPLC using a Tosoh TSK gel Super SW2000 gel filtration column.

Further, 22% greater target protein is observed in the extraction stage in the presence of arginine compared to the process not involving arginine in the extraction stage (Table 7).

After cationic exchange chromatography, the % recovery of IFNβ 1b obtained in the presence of arginine is at least 23% greater than the protein recovered in the absence of arginine. The level of purity of IFNβ 1b is also considerably higher when extracted in the presence of arginine (Table 8).

In another embodiment, the yield of recombinant interferon-β 1b is in the range of 0.5 to 5 grams/liter. A direct scale up from 8.6 gram cell mass to 600 gm was observed to be reproducible.

The present process for the purification of IFN-β 1b described in the above embodiments can be employed in the purification and recovery of recombinant proteins, more specifically those recombinant proteins that are sequestered in inclusion bodies in the host cell.

Therefore, in one embodiment, the present disclosure provides a process for the recovery and purification of recombinant insulin and analogues thereof from bacterial inclusion bodies comprising,
i. lysing host cells containing said protein to obtain a cell lysate followed by centrifuging the cell lysate to obtain an insoluble fraction,
ii. subjecting the said insoluble fraction to treatment with an extraction solution comprising; a) at least one detergent having alkaline pH, b) at least one chaotropic agent having alkaline pH, c) at least one cosmotropic agent and d) at least one protein folding agent viz. arginine, wherein, the extraction solution has a pH of around 1.5 to around 12, to obtain a solubilized fraction,
iii. allowing the solubilized fraction to mature followed by addition of an oxidizing agent to the extraction mixture, and
iv. filtering the extract and subjecting it to ion exchange chromatography followed by ultrafiltration and diafiltration to obtain an extract comprising recombinant protein in soluble form, in high yield with high % purity.

Arginine shows a positive impact in minimizing the aggregates during solubilization and refolding of pro-insulin and expectedly does the same with insulin analogues and related class of proteins. The effect of arginine during the solubilization and refolding of pro-insulin was observed. An increase in the % purity at the pro-insulin refolding stage was observed to be ranging from about 60% to about 70%. A further increase in the % purity at the final purification stage of chromatography was observed to be ranging from about 85% to 95%. A consequent increase in the yield of the final pro-insulin in the functional configuration is obtained in the concentrations ranging from about 2000 mg/liter to around 3200 mg/liter. (Table 2 describes the effect of arginine on the pro-insulin in the presence and absence of arginine at different concentrations.

In presence of arginine an increase in the pro-insulin yield by ˜9% and yield by ˜17% is observed. L-Arginine is found effective when used in combination with that of mild conditions like nonionic/zwitterionic detergents and basic pH.

In a preferred embodiment, the present disclosure provides a kit for recovery and purification of proteins from bacterial inclusion bodies, comprising:
(i) an extraction solution for dissolving said proteins, comprising at least one detergent having alkaline pH; at least one chaotropic agent having alkaline pH; at least one cosmotropic agent; and arginine; and
(ii) an oxidizing agent is selected from a thiol, a disulfide, or a mixture thereof for oxidizing said dissolved proteins;
   wherein said extraction solution has a pH of between about 1.5 and about 12.

Advantageously, the protein recovery and purification kit described above is used for extraction and purification of proteins expressed in bacterial inclusion bodies by the process disclosed herein. Industrial fermentation processes may utilize the present recovery and purification kit and the process described herein to obtain recombinant proteins in enhanced yield and high purity levels for therapeutic applications.

### Examples

Following examples are given by way of illustration therefore should not be construed to limit the scope of the invention.

### Example 1(A): Expression of Recombinant Pro-Insulin in E. coli BL21(DE3)

Pro-insulin was expressed in E. coli BL21(DE3), using IPTG (Iso Propyl Thio Galactoside) as inducer at an order of 10 gm/lit fermentation broth.

### Example 1(B): Expression of Recombinant Pro-Insulin in Pseudomonas

Pro-insulin was expressed in Pseudomonas fluorescens using IPTG (Iso Propyl Thio Galactoside) as inducer at an order of 10 gm/lit fermentation broth.

### Example 2: Recovery and Purification of Pro-Insulin and its Analogues

E. coli cells containing inclusion bodies enriched with insulin were lysed using a High Pressure Homogenizer at 1000 bar in the presence of a lysis buffer containing 20 mM basic buffer Tris, pH 8. The cell lysate was subjected to centrifugation to obtain an insoluble pellet comprising the inclusion bodies. Removal of contaminant proteins present in the inclusion bodies was accomplished via two sequential washes with 20 mM Tris/Triton X-100 buffer with detergent, pH8. This insoluble fraction of pro-insulin was subjected to treatment with an extraction buffer (20 mM buffer Tris, 2M cosmotrope, i.e. sodium chloride, potassium chloride, ammonium sulfate, and mixtures thereof, 2M Urea, 1% an amphoteric detergent i.e. sulfobetaines, 0.5M L-Arginine). Accordingly, the concentration of the lysis, extraction and maturation buffer are provided herein below in Table 1:

TABLE 1
Buffers and approximate volumes for Lysis, Extraction and Maturation

| Phase | Solution | Volume (Condition) |
|---|---|---|
| Lysis | 20 mM Tris, pH 8.0 | 0.5 L |
| Wash | 20 mM Tris, pH 8.0 | 0.5 L |
| Extraction | 20 mM Tris, Cosmotrope: 2M NaCl, | 0.7 L |
| | 2M Urea, 1% amphoteric detergent, | |
| | i.e. sulfobetaines, 0.5M Arginine pH | |
| | 8.0 | |
| Conditioning | 5 mM Tris pH 8.5 | 28 L |
| Diluent | | |

Extraction of pro-insulin from the washed inclusion bodies with the extraction buffer was performed at pH 12. Pro-insulin was subjected to treatment with 2 sets of extraction buffers with different dilutions. 500 mM in a 1:6 dilution (1 gm inclusion bodies and 6 ml of extraction buffer), 125 mM in a dilution of 1:24 (1 gm inclusion bodies and 24 ml of extraction buffer). The solution comprising the pellet and the extraction buffer was stirred continuously for one hour. This mixture was centrifuged at 12,000 RCF. The supernatant was retained and reverse diluted using a peristaltic pump for one hour with continuous mixing. The pH was adjusted to around 7.2 using NaOH. The solution containing solubilized insulin was allowed to mature. The solubilized protein solution was then subjected to immobilized affinity chromatography technique (IMAC) (FIG. 1 depicts the % purity of pro-insulin solubilized and refolded in the presence of arginine).

### Example 3: Recovery and Purification of Pro-Insulin and its Analogues

Insulin was recovered using an extraction solution (5 mM basic buffer Tris, 0.5M L-Arginine, 10 mM reducing agent Cysteine). E. coli cells containing inclusion bodies enriched with insulin were lysed using a High Pressure Homogenizer at 1000 bar in the presence of a lysis buffer containing 20 mM Tris, pH 8. The cell lysate was subjected to centrifugation to obtain an insoluble pellet comprising the inclusion bodies. Removal of contaminant proteins present in the inclusion bodies was accomplished via two sequential washes with 20 mM Tris/Triton X-100 buffer containing detergent, pH8. This insoluble fraction of pro-insulin was subjected to treatment with the extraction solution. Extraction of pro-insulin from the washed inclusion bodies was performed at pH 12 to 12.2 for 5 to 10 minutes, followed by addition of 10 mM reducing agent β-mercaptoethanol. Pro-insulin was subjected to treatment with 2 sets of extraction solutions with different dilutions: 500 mM in a 1:6 dilution (1 gm inclusion bodies and 6 ml of extraction buffer), 125 mM in a dilution of 1:24 (1 gm inclusion bodies and 24 ml of extraction buffer). The solution comprising the pellet and the extraction solution was stirred continuously for one hour. This mixture was centrifuged at 12,000 RCF. The supernatant was retained and reverse diluted using a peristaltic pump for one hour with continuous mixing. The pH was adjusted to around 7.2 using NaOH. The solution containing solubilized insulin was allowed to mature. The solubilized protein solution was then subjected to immobilized affinity chromatography technique (IMAC).

L-arginine was found to be effective when used in presence of mild conditions, of pH 7 and non-ionic or zwitterionic detergents. The process employed in Examples 2 and 3 benefits higher quality and increased yield of pro-insulin without exposing proteins to harsh conditions posed by anionic detergents, and organic solvent extractions.

### Example 3: Extraction of Pro-Insulin in the Absence of Protein Folding Agent

The solubilization buffer used for the extraction of insulin was 5 mM buffer Tris pH, 10 mM reducing agent-like Cysteine, and 10 mM reducing agent-2 β-mercaptoethanol with pH 10.75±0.25. 1 gm wet weight of inclusion body (IB) was solubilized in 6 ml of buffer (1:6 ratio) and pH of solubilized protein was adjusted to 11.8 and incubated for 5-10 minutes upon stirring and the pH was brought down to 10.75±0.25 and was incubated for 1 hr under stirring conditions. At the refolding stage, buffer comprising 5 mM buffer Tris, pH 10.75+0.25 was used. Refolding is performed by simple dilution of protein solution with buffer in a 1:30 ratio in duration of 10 minutes followed by stirring of solution for 15 minutes more. The stirred solution is incubated for 16-20 hours at 2 to 8° C. in a static condition (FIG. 2 depicts % purity of pro-insulin solubilized and refolded in absence of arginine).

Extraction of 1 gm wet weight of inclusion bodies solubilized in 24 ml of extraction buffer was also used for recovery and purification of protein. Results are reported in Table 7.

### Example 4: Effect of the Extraction Solution Comprising Arginine on Yield and Purity of Pro Insulin

The procedure of Example 3 was repeated, except that the solubilization buffer used for the extraction of insulin was 5 mM buffer Tris pH, 10 mM reducing agent-01Cysteine, 10 mM reducing agent-02 β-mercaptoethanol, and 0.5M arginine, with a pH 10.75±0.25. According to the procedure, 1 gm wet weight of inclusion body (IB) was solubilized in either 6 ml of buffer (1:6 ratio) or 24 ml of buffer (1:24 ratio). Results are reported in Table 2.

As shown in Table 2, use of an arginine-free solubilization buffer at a 1:6 ratio produced a proinsulin product having 37.7% purity at the refolding step. Addition of arginine to the solubilization buffer produced a product having 64.2% purity at the refolding step. After refolding, the product obtained by solubilization in the absence of arginine had a purity of 85%, while the product obtained by solubilization in the presence of arginine had 89% purity.

Similarly, use of an arginine-free solubilization buffer at a 1:24 ratio produced a proinsulin product having 54.9% purity at the refolding step (FIG. 2(a)). Addition of arginine to the solubilization buffer produced a product having 64% purity prior to the refolding step (FIG. 1(a)). After refolding, the product obtained by solubilization in an arginine-free solubilization buffer at a 1:24 ratio had a purity of 85% (FIG. 2(b)), while the product obtained by solubilization in the presence of arginine had 89.9% purity (FIG. 1(b)).

Extraction of Proinsulin in presence of arginine at 0.5M in the extraction buffer showed minimized aggregate formation during solubilization and refolding of protein. An increase in yield of proinsulin by approximately 9% to 17% was observed.

TABLE 2
Effect of Arginine in Solubilization and Refolding of Insulin:

| | | Purity at | Purity at | Yield |
|---|---|---|---|---|
| S. | | Refolding, | IMAC | (IMAC Run), |
| No | Parameter | % | step, % | mg/lit |
| 1. | Without Arginine, | 37.7 | 85 | 1979 |
| | Solubilization at 1:6 ratio | | | |
| 2. | Arginine, Solubilization at | 64.2 | 89 | 1999 |
| | 1:6 ratio | | | |
| 3. | Without Arginine, | 54.9 | 85 | 2635 |
| | Solubilization at 1:24 ratio | | | |
| 4. | With Arginine, | 64 | 89.9 | 3164 |
| | Solubilization at 1:24 ratio | | | |

(IMAC: Immobilized Metal Ion Chromatography)

### Example 5(A): Expression of Recombinant Interferon β (rIFN-β) in E. coli BL21 (DE3)

Interferon β-1b was expressed in E. coli BL21(DE3), using IPTG (Iso Propyl Thio Galactoside) as inducer at an order of 10 gm/lit fermentation broth. Crude IFN-β-1b was expressed and distributed as both soluble and insoluble form, predominantly insoluble form.

### Example 5(B): Expression of Recombinant Interferon β (rIFN-β) in Pseudomonas Species

Interferon β-1b was expressed in Pseudomonas fluorescens using IPTG (Iso Propyl Thio Galactoside) as inducer at an order of 10 gm/lit fermentation broth. Crude IFN-β-1b was expressed and distributed as both soluble and insoluble form, predominantly insoluble form.

### Example 6: Lysis, Extraction and Maturation

Depending on the fermentation productivity and biomass (titer), approximately 100 g of frozen cell paste was re-suspended at 20% solids into Tris buffer and lysed by High Pressure homogenizer at 1000 bar. Batch centrifugation of the cell lysate yielded a soluble fraction in the form of a supernatant and a pelleted portion comprising the insoluble IFN-3. IFN-β is generally found sequestered in inclusion bodies in the host cell, therefore it is considered to be present in the pellet form. The pellet was retained for further extraction procedures and the supernatant was discarded. The IFN-1β containing pellet was re-suspended and was washed in Tris buffer, and once again centrifuged. The washed IFN-1 β pellet amounting to at least 24 gm was stored at −20° C.

The washed IFN-1 β pellet was subjected to treatment with an extraction solution comprising a buffer, at least one detergent having alkaline pH, at least one chaotropic agent having alkaline pH, at least one cosmotropic agent, at least one protein folding agent, at pH8. The chaotropic agent used is urea, the detergent used is an amphoteric detergent selected from sulfobetaines, cosmotropic agent is selected from NaCl, KCl or (NH₄)₂SO₄. IFN-1 β is extracted into a solution in the solubilized form. Around 70 ml of the extraction buffer for each gm of IFN-1 β by wet weight is added to the washed pellet. The pH is adjusted to 8 using 2N HCl/NaOH. The solution obtained containing the solubilized IFN-1 β was stirred on a magnetic stirrer continuously for 1 hr followed by centrifuging the extract at 12000 RCF. The extract supernatant obtained was reverse diluted at 40× using peristaltic pump for an hour time under continuous mixing. The pH was adjusted to 8.5 using 2N NaOH, if necessary. The solution was matured for 16-18 hrs followed by addition of 25 μM L-Cystine to the matured solution and was incubated for 1 hr. Adjust the pH to 7.2 using 2N HCl.

In a preferred aspect, buffers and approximate volumes for Lysis, Extraction and Maturation are provided herein below in table 3.

TABLE 3
Buffers and approximate volumes for Lysis, Extraction and Maturation

| | | Volume |
|---|---|---|
| Phase | Solution | (Condition) |
| Lysis | 20 mM Tris, pH 8.0 | 0.5 L |
| Wash | 20 mM Tris, pH 8.0 | 0.5 L |
| Extraction | 20 mM Tris, Cosmotrope: 2M NaCl, 2M | 0.7 L |
| | Urea, amphoteric detergent | |
| | (sulfobetaines): 1%, 0.5M Arginine pH | |
| | 8.0 | |
| Conditioning | 5 mM Tris pH 8.5 | 28 L |
| Diluent | | |

### Example 7: Filtration and Column Chromatography

After maturation, the solution is filtered through 0.45 μm membrane then loaded onto a cation exchange column, i.e. Capto SP impRes column and subjected to cation exchange chromatography (GE Healthcare, P/N: 17-5468-03). Column Dimensions (D×H, volume): 5 cm×13 cm, 255 ml. The cation exchange chromatogram is depicted in FIG. 4.

Subsequent to cation exchange chromatography, a recovered sample may be loaded onto an anion exchange column and subjected to anion exchange chromatography.

TABLE 4
Capto SP impRes Cation Exchange Column: phases of operation

| | | | Flow rate | Flow |
|---|---|---|---|---|
| Phase | Solution | Volume | (cm/hr) | direction |
| Pre-equilibration | 20 mM buffer HEPES + 0.5M | 2 CV | 400 | Down flow |
| | Urea + 0.25M cosmotrope: | | | |
| | NaCl + 0.05% detergent | | | |
| | sulfobetaines pH 7.2 | | | |
| Equilibration | 20 mM buffer HEPES + 0.5M | 4 CV | 400 | Down flow |
| | Urea + 0.05% sulfobetaines, | | | |
| | pH 7.2 | | | |
| Load | 40X diluted refolded sample | 1.5 mg/ml | 400 | Down flow |
| | | resin | | |
| Wash | 20 mM buffer HEPES + 0.5M | 4 CV | 400 | Down flow |
| | Urea + 0.05% sulfobetaines | | | |
| | detergent, pH 7.2 | | | |
| Elution | 10 CV Gradient: 20 mM buffer | 10 CV | 100 | Down flow |
| | HEPES, 0.5M urea, pH 7.2 | | | |
| | with cosmotrope NaCl in the | | | |
| | range of 150-250 mM | | | |
| Regeneration | 0.5N NaOH + cosmotrope 1M | 2 CV | 400 | Down flow |
| | NaCl | | | |
| Storage | 20% Ethanol | 2 CV | 400 | Down flow |

Capto SP impRes Chromatogram (1913 mg total protein (measured by _{(A280)}loaded on 255 ml column resin).

### Example 8

### Ultrafiltration and Diafiltration

The eluate obtained after passing through a column was directly collected in a 10 mM acidic buffer (150 ml), having pH ranging from 2 to 5 while mixing the solution. Accordingly, the acid buffers are selected from sodium acetate, acetic acid, mostly having a pH of about 4. Upon completion of the elution process, volume of the eluted extract is made up with acetate buffer to give a final 10 fold elution volume. The final extract was loaded on to Sartocon slice 200 cm², 5 kD UFDF cassette. The diluted retentate was then concentrated 10-fold and diafiltered for 10 DVs. The retentate was then concentrated to minimum working volume (25 ml). Upon completion of the run flush the cassette with 22 ml of acidic buffer, the retentate was pooled and washed. (99Membrane: Sartocon slice 200, Catalog No-1442902E-SW, Cutoff: 5 KD, Area: 200 cm²

Operational conditions: TMP—17.6 psi, Flux—24LMH, Process time—3 hrs.

TABLE 5
UFDF Operation

| Phase | Solution | No. of Dia volumes |
|---|---|---|
| Initial Dilution | 10 mM acidic buffer pH 3.8 | 10 DV |
| Diafiltration | 10 mM acidic buffer pH 3.8 | 10 DV |

TABLE 6
% Recovery and % Purity

| | Total | | % Purity | |
|---|---|---|---|---|
| | Protein | % Step | by RP | % Purity |
| Step | (mg) | Recovery | HPLC | by SEC |
| Lysis (34 gm cell paste, as | 730 | — | — | — |
| per 6.1 gm/lit harvest) | | | | |
| After cation exchange | 248 | 33.9 | 92.60 | — |
| chromatography | | | | |
| After anion exchange | 205.6 | 82.9 | 94.47 | 96.21 |
| chromatography | | | | |
| UFDF Retentate + wash | 190.6 | 92.17 | 95.60 | 97.17 |

TABLE 7
Comparative analysis of % IFNβ 1b recovery

| | (improved process | | (Without L- | |
|---|---|---|---|---|
| | with L-Arginine) | Recovery, | Arginine) | Recovery, |
| Step | Recovery, mg | % | Recovery, mg | % |
| Lysis | 6110 | 100% | 6110 | 100% |
| Extraction | 4820.2 | 78.89% | 3468.6 | 56.77% |

TABLE 8

| | With | | Without L- | |
|---|---|---|---|---|
| | L-Arginine | | Arginine | |
| | Recovery, | Recovery, | Recovery, | Recovery, |
| Step | mg | % | mg | % |
| CEX | 1229.6 | 25.5 | 76.3 | 2.2 |

| | | |
|---|---|---|
| (Chromatography-I) | Purity - 94% | Purity - 75% |

1. An extraction solution for recovery of proteins from bacterial inclusion bodies, comprising:
at least one detergent;
at least one chaotropic agent;
at least one cosmotropic agent; and
a protein folding agent;
wherein said extraction solution has a pH of between about 1.5 and about 12.

2. The extraction solution of claim 1, wherein said extraction solution has a pH of between about 7.2 and about 8.5.

3. The extraction solution of claim 1, wherein said chaotropic agent is selected from the group consisting of urea, guanidinium chloride, thiourea, and mixtures thereof.

4. The extraction solution of claim 1, wherein said cosmotropic agent is selected from the group consisting of chloride salts, sulfate salts, and mixtures thereof.

5. The extraction solution of claim 1, wherein said cosmotropic agent is selected from the group consisting of sodium chloride, potassium chloride, ammonium sulfate, and mixtures thereof.

6. The extraction solution of claim 1, wherein said protein folding agent is arginine.

7. A kit for recovery of proteins from bacterial inclusion bodies, comprising:
an extraction solution for dissolving said proteins, comprising at least one detergent; at least one chaotropic agent; at least one cosmotropic agent; and arginine; and
an oxidizing agent for oxidizing said dissolved proteins;
wherein said extraction solution has a pH of between about 7.2 and about 8.5; and
wherein said oxidizing agent is a thiol, a disulfide, or a mixture thereof.

8. A process for recovery of a protein from bacterial inclusion bodies using an extraction solution comprising at least one detergent; at least one chaotropic agent; at least one cosmotropic agent; and a protein folding agent; wherein said extraction solution has a pH of between about 1.5 and about 12,
the process comprising:
i. lysing bacterial host cells containing said protein to obtain a cell lysate;
ii. centrifuging the cell lysate to obtain an insoluble fraction;
iii. extracting the insoluble fraction with said extraction solution to obtain an extraction mixture, and allowing the extraction mixture to mature;
iv. adding an oxidizing agent to the extraction mixture; and
v. subjecting the extraction mixture to ion exchange chromatography and at least one of ultrafiltration and diafiltration to obtain an extract comprising said protein in a soluble form.

9. A process for recovery of a protein from bacterial inclusion bodies using the kit of claim 7, comprising:
i. lysing bacterial host cells containing said protein to obtain a cell lysate;
ii. centrifuging the cell lysate to obtain an insoluble fraction;
iii. extracting the insoluble fraction with said extraction solution to obtain an extraction mixture, and allowing the extraction mixture to mature;
iv. adding said oxidizing agent to the extraction mixture; and
v. subjecting the extraction mixture to ion exchange chromatography and at least one of ultrafiltration and diafiltration to obtain an extract comprising said protein in a soluble form.

10. The process of claim 8, wherein said chaotropic agent is selected from the group consisting of urea, guanidinium chloride, thiourea, and mixtures thereof.

11. The process of claim 8, wherein said cosmotropic agent is selected from the group consisting of sodium chloride, potassium chloride, ammonium sulfate, and mixtures thereof.

12. The process of claim 8, wherein said protein folding agent is arginine.

13. The process of claim 8, wherein said protein is a recombinant protein.

14. The process of claim 13, wherein the recombinant protein is a recombinant interferon, a recombinant insulin, a recombinant insulin-like growth factor binding protein.

15. The process of claim 13, wherein:
the recombinant protein is a recombinant interferon; and
the extraction solution has a pH of between about 7.2 and about 8.5.

16. The process of claim 15, wherein the recombinant interferon is selected from the group consisting of interferon-β, interferon-β 1b, interferon-α, interferon-κ, interferon-τ, and interferon-ω.

17. The process of claim 13, wherein:
the recombinant protein is a recombinant insulin; and
the extraction solution has a pH of between about 1.5 and about 12.

18. The process of claim 17, wherein the recombinant insulin is an insulin analogue selected from the group consisting of glargine insulin, lispro insulin, and aspart insulin.

19. The process of claim 13, wherein:
the recombinant protein is a recombinant proinsulin fusion protein.

20. The process of claim 19, wherein the recombinant proinsulin fusion protein is a fusion protein comprising a proinsulin peptide chain linked to a second biologically active peptide chain.

21. The process of claim 19, wherein the recombinant proinsulin fusion protein is a fusion protein comprising a proinsulin peptide chain linked to a biologically active peptide chain selected from the group consisting of transferrin, interleukin-2, and glutathione-S-oxidase.

22. The process of claim 8, wherein the oxidizing agent is selected from the group consisting of a thiol, a disulfide, and mixtures thereof.

23. The process of claim 8, wherein the oxidizing agent is selected from the group consisting of cystine, cysteine, oxidized glutathione, reduced glutathione, dithiothreitol, and mixtures thereof.

24. The process of claim 8, wherein said subjecting the extraction mixture to ion exchange chromatography comprises sequentially subjecting the extraction mixture to cation exchange chromatography and to anion exchange chromatography.

25. The process of claim 8, wherein said subjecting the extraction mixture to ion exchange chromatography comprises subjecting the extraction mixture to at least one of:
cation exchange chromatography over a cation exchange resin selected from the group consisting of sepharose, cross-linked poly[styrene-co-divinylbenzene] with a polyhydroxyl surface coating, and high flow agarose; and
anion exchange chromatography over an anion exchange resin having positively charged groups.

26. A process for purification of a recombinant protein, comprising:
i. lysing bacterial host cells containing expressed recombinant protein to obtain a cell lysate;
ii. centrifuging the cell lysate to obtain an insoluble fraction;
iii. extracting the insoluble fraction with an extraction solution comprising arginine to obtain an extraction mixture, and allowing the extraction mixture to mature;
iv. adding an oxidizing agent to the extraction mixture; and
v. subjecting the extraction mixture to ion exchange chromatography and at least one of ultrafiltration and diafiltration to obtain an extract comprising said recombinant type 1 interferon in a soluble form;
wherein said recombinant protein is a recombinant interferon, a recombinant insulin, or a recombinant insulin-like growth factor binding protein.

27. The process of claim 23, wherein said recombinant type 1 interferon is recovered in a yield of between 50% and 95%.

28. The process of claim 23, wherein said recombinant type 1 interferon is recovered in a purity of between 92% and 97%.
